(19) United States
(12) Patent Application Publication
(10) Pub. No.: US 2020/0093687 A1
(43) Pub. Date: Mar. 26, 2020

(54) SYSTEMS AND METHODS FOR BIOMODULATION USING A FLUID IMMERSION PATHWAY AND PHOTO-INDUCED COHERENT RESONANCE ENERGY TRANSFER

(71) Applicant: VARUNA BIOMEDICAL CORPORATION, Incline Village, NV (US)

(72) Inventor: Stanton L. THOMAS, Incline Village, NV (US)

(21) Appl. No.: 16/471,663

(22) PCT Filed: Jan. 12, 2018
(86) PCT No.: PCT/US18/13543

§ 371 Date: Jun. 20, 2019

Related U.S. Application Data
(60) Provisional application No. 62/445,631, filed on Jan. 12, 2017.

(51) Int. Cl. A61H 33/00; A61N 5/06

(52) U.S. Cl. CPC A61H 33/6005; A61N 5/0613; A61H 2201/10; A61N 2005/067; A61N 2005/0652; A61N 2005/0668; A61N 2005/064

(57) ABSTRACT

Systems and methods for biomodulation of a target volume of tissues within a living subject are provided. A biomodulation system can include a number of features, including a light-emitting system to produce an elevated molecular excitation state and coherent resonance energy transfer within a fluid volume, a fluid container configured to hold a fluid and the living subject to be treated, and a fluid management system of the fluid volume. The biomodulation system can implement a treatment regime involving variable control parameters including wavelength, irradiance, energy density, pulse frequency, peak power, irradiation time and others to affect biomodulation depending upon the characteristics and condition of a target volume within the living subject.

### CROSS REFERENCE TO RELATED APPLICATIONS

This application claims the benefit of priority to U.S. Provisional Application No. 62/445,631, filed Jan. 12, 2017, which is herein incorporated by reference.

This application is related to U.S. application Ser. No. 14/930,516, titled “Systems and Methods for Detecting and Visualizing Biofields with Nuclear Magnetic Resonance Imaging and QED Quantum Coherent Fluid Immersion”, filed Nov. 2, 2015.

### INCORPORATION BY REFERENCE

All publications and patent applications mentioned in this specification are herein incorporated by reference to the same extent as if each individual publication or patent application was specifically and individually indicated to be incorporated by reference.

### FIELD

This disclosure generally relates to low-level light-based biomodulation therapies used to treat and prevent injury, disease, and disorder in living organisms. More specifically, this disclosure provides systems and methods for biomodulation therapy using fluid immersion and photo-induced coherent resonance energy transfer.

### BACKGROUND

As lasers were first developed in the early 1960s, the promise of light-based therapeutic modalities to revolutionize the medical field was almost immediately evident. Applications in dermatology and ophthalmology were among the first to be developed, followed later by therapeutic pain relief, wound healing, and treatment of neurological disorders. Two general mechanisms of action of photonic energy have been harnessed to create new modalities; photothermal (high energy action causing heating, burning, and cauterizing of tissues) and photodynamic (low energy action causing chemical changes to tissues).

The growing application of low level dosages of photonic energy using low power laser or LED devices to target specific chromophores (e.g., melanin, collagen, hemoglobin, and water) for the purpose of modulating or activating regenerative, reparative and protective processes within the tissues of living organisms has been referenced by medical practitioners and researchers using a number of general terms. These terms include Low Level Light Therapy (LLLT), Low Power Light Therapy (LPLT), Light Emitting Diode Therapy (LEDT), Low Energy Photon Therapy (LEPT), and others. In practice these terms have been considered by many to be overly general and vague.

In a recent paper by J Anders et al. a more accurate term and comprehensive definition was submitted for inclusion in the Medical Subject Headings (MeSH) controlled vocabulary thesaurus; Photobiomodulation (PBM) Therapy. This term is defined as, “A form of light therapy that utilizes non-ionizing forms of light sources, including lasers, LEDs, and broadband light, in the visible and infrared spectrum. It is a non-thermal process involving endogenous chromophores eliciting photophysical (i.e., linear and nonlinear) and photochemical events at various biological scales. This process results in beneficial therapeutic outcomes including but not limited to the alleviation of pain or inflammation, immunomodulation, and promotion of wound healing and tissue regeneration.”

While the specific mechanisms of PBM-based therapy do not yet appear to be fully understood or articulated, the generally-accepted primary action pathway at the molecular, cellular, and tissue levels is related to the absorption of infrared light by the chromophore

Cytochrome c Oxidase (CcO) in the mitochondrial electron transport chain. CcO is one of four transmembrane proteins of the inner mitochondrial membrane, and the absorption of photons by CcO stimulates activity in the electron transport chain resulting in increased adenosine triphosphate (ATP) production, with a corresponding increase in oxygen consumption. The change in oxidation-reduction state coupled with increased ATP production that provides greater biochemical energy is believed to drive a number of secondary and tertiary events including gene transcription, protein synthesis and the production of reactive oxygen species (ROS). The redox changes are also thought to drive the displacement of nitric oxide (NO), which in turn stimulates blood flow and electron transport chain activity. Light is also believed to induce upregulation and downregulation of multiple genes in both the nucleus and mitochondria resulting in expression for cell proliferation, survival, antioxidant production, transcription, and growth factor production.

While the specific mechanisms of PBM-based therapy are not fully understood, the generally-accepted primary action pathway at the molecular, cellular, and tissue levels is related to the absorption of light by the chromophore Cytochrome c Oxidase (CcO) in the mitochondrial respiratory chain. The mitochondrial structures within cells are often referred to as the cellular power-houses, and CcO is the transmembrane protein of the inner mitochondrial membrane. The absorption of photons by CcO stimulates activity in the electron transport chain resulting in increased adenosine triphosphate (ATP) production, with a corresponding increase in oxygen consumption. The change in oxidation-reduction state is believed to drive a number of secondary and tertiary events including gene transcription, protein synthesis and the production of reactive oxygen species (ROS). The redox changes are also thought to drive the displacement of nitric oxide (NO), which in turn stimulates blood flow and electron transport chain activity. Light is believed to induce upregulation and downregulation of multiple genes in both the nucleus and mitochondria resulting in expression for cell proliferation, survival, antioxidant production, transcription, and growth factor production.

While the US FDA has cleared the use of light-based therapies for the relief of pain-related symptoms from ailments such as carpel tunnel, muscle spasms, arthritis and others, the broader clinical and therapeutic reach of PBM is becoming more evident. The reparative, regenerative, and protective effects of PBM-based therapeutic modalities across a diverse array of diseases and disorders have been studied and documented with increasing focus over the past decade. These studies, involving both animal models and human subjects, point to the potential of PBM as a multi-target therapy for complex diseases based upon its capacity to stimulate metabolic energy pathways in a variety of tissues.

In the years since its initial development, dermatology has been perhaps the most prolific area of clinical development for PBM applications due to the abundance of chromophores in the skin and the high accessibility of photonic energy to superficial dermal layers. PBM has been shown to have beneficial effects on wrinkles, scaring, and burns, as well as demonstrating the capacity to trigger protective action again UV-induced skin damage. It has been used to treat pigmentary disorders, and inflammatory diseases such as acne and psoriasis.

Another area of focus has been ophthalmology where application of PBM-based therapies has shown promise in treating a variety of retinal disorders. Because the retina contains neurons with high energy demands that rely on mitochondria-produced ATP, it seems intuitive that any mechanism that counteracts mitochondrial dysfunction and stimulates ATP synthesis may likely provide therapeutic benefit in relation to retinal disorders. Indeed, studies have demonstrated the potential for PBM to arrest and even reverse retinal damage associated with age-related macular degeneration (AMD). In both animal and human models, PBM has been shown to produce positive results in treating retinopathy associated with diabetes, premature birth, and methanol intoxication. There is some evidence that PBM may be used to treat retinitis pigmentosa (RP), an inherited disease featuring retinal degeneration.

In the field of neurology, PBM is being studied within the context of the three broad classes of brain disorders including traumatic injury (Traumatic Brain Injury, stroke, etc.), neurodegenerative disease (Alzheimer's, Parkinson's, Dementia), and psychiatric disorders (PTSD, anxiety and depression). In Alzheimer's and Parkinson's studies using animal models (transgenic mice and non-human primates), as well as limited human clinical studies, results show neurogenerative, neuroprotective, synaptogenerative, vascular-reparative effects capable of slowing and arresting disease progression. These studies also showed improved executive function in animals, as well as improvements executive, cognitive and emotional functions in humans. The now-famous NEST study showed promising results after a single treatment in acute stroke patients, before that study was terminated in a later stage. A number of TBI-related studies using both animal models and humans have demonstrated significant improvements in executive function, neurobehavioral, memory, and learning test results after PBM treatment. In PTSD studies, results showed improved sleep patterns and fewer symptoms.

PBM therapies have demonstrated interesting results in an area related to brain health: memory loss and mood disorders. It has been demonstrated that severe depression and

PTSD are associated with decrease metabolic activity in the prefrontal areas of the brain. PBM's ability to stimulate metabolic energy pathways may be the driver behind demonstrated improvements in performance in studies related to memory, cognitive, and emotional disorders.

Light-based therapeutic modalities have been shown to have immune-modulating effects in studies involving CAT hypothyroidism, allergic rhinitis, tetanus, herpes simplex, and psoriasis. Significant improvements in immunological indices are reported in PBM studies involving both animal models and humans, including enhanced lymphocyte proliferation and antibody profiles. PBM treatment has also been demonstrated to boost the immune response when administered in combination with immunogenic agents. A recent study of the effects of PBM on human monocyte polarization into M1 macrophages suggests the potential for light-based therapies to promote immunity to viral infections, tumors, and allergic diseases.

In the study of musculoskeletal disease and disorder, PBM has been used to enhance performance and accelerate recovery of exercise-related functional and biochemical markers. The therapy has demonstrated positive results in application to tendinopathies and muscle injuries. The protective and restorative effects of PBM on muscle tissue appear to retard the progression of dystrophies when used preventatively. Using animal models, researchers have shown PBM to be potentially effective in treating Duchene muscular dystrophy (DMD). Treatments demonstrated improved regenerative capacity, promoting muscle cell proliferation while decreasing inflammatory response and oxidative stress in dystrophic muscle cells.

In studies of light-based treatment modalities for cardiovascular disease conducted mainly in Eastern Europe, PBM has been shown to improve cardiac performance and myocardial contractibility while having anti-anginal and antihypertensive effects. The results have been accompanied by positive changes in lipid metabolism, antioxidant defense, and microcirculation, and increases in myocardial, coronary and aerobic reserves. In studies involving animal models, PBM therapy has significantly reduced the size of infarct in cases of acute myocardial infarction (MI). Therapy administered post-operatively has shown the potential of PBM to decrease cardiac cellular damage and help accelerate the repair of cardiac tissues.

PBM has demonstrated effectiveness in combating pulmonary diseases such as chronic obstructive pulmonary diseases (COPD, incl. bronchitis and emphysema), acute respiratory distress syndrome (ARDS), and asthma. In animal model studies (mice, rats) involving LPS-induced pulmonary and extra-pulmonary ARDS, PBM reduced acute pulmonary inflammation independent of the primary cause of the condition by preventing the migration of neutrophils and pro-inflammatory mediators to the lungs. Another study investigated the potential for an adjuvant therapy to augment stem cell activity in combating COPD, which is believed to be inflammatory- and/or immunologically-mediated. The study concluded that the ability of PBM to induce growth factor production, inhibit inflammation, and stimulate vascular development and stem cell proliferation, make it a potentially important new modality in combination with regenerative medicine.

Finally, the results of in vitro and in vivo studies involving both animal models and humans suggest that PBM has the potential to be a multi-target anti-tumor therapy, including the ability to reduce the adverse side-effects associated with chemotherapy. It is thought that PBM's ability to stimulate the cellular respiratory chain and ATP production promotes selective cellular apoptosis and differentiation, thus slowing and possibly arresting tumor proliferation as evidenced by tumor-volume reduction. The anti-inflammatory and immune-boosting effects of PBM are also thought to play a major role in its anti-tumor capacity.

The vast majority of PBM studies have demonstrated not only positive results, but that this therapeutic approach is also clinically safe showing no apparent dose-toxicity or side-effects. Specific studies assessing the risk of carcinogenesis after many treatments with non-ablative lasers, flash lamps, intense pulsed light have failed to obtain evidence of skin toxicity. The main precaution employed in human studies is the use of protective eye-ware and careful modulation of output energy, particularly when using laser devices. When viewed in comparison to drug-based therapies, whose often transitory results are accompanied by cytotoxicity and physical and psychological side effects, PBM appears to offer both a safe and cost-effective alternative approach.

As published research continues to point to the potential benefit of PBM-based therapies across of broad spectrum of disease and disorders, there has begun a corresponding proliferation of research and commercial therapeutic device technologies entering the fledgling market. These devices fall into two general categories, including lasers and light-emitting diodes (LEDs). Lasers provide monochromatic, directional, and coherent EM radiation with a constant beam width which allows for the delivery of concentrated energy to greater depth. LEDs on the other hand, are non-coherent and produce most of their light in a narrow range of wavelengths, and therefore deliver less concentrated energy and produce less heat. Regardless of their differences, both low-level light-based technologies have demonstrated significant positive results. Innovative device designs allow for a diverse array of use modalities including surgical implant, intravenous, intranasal, hand held, wearable patch or wrap, helmet, lamp, and whole body light pod.

Despite its apparent promise there are still challenges and some controversy facing PBM-based therapy. One of the main challenges relates to sometimes inconsistent and/or unreproducible results. PMB results are dependent upon a number of specific parameters in combination, including output power, energy density, pulse frequency and duration, wavelength, contact modality, input light source, and biochemical properties of the target tissue volume. At the present stage of this field, there is a lack of standardization of parameters and researchers don't always specify a comprehensive set of relevant parameter values employed. PBM is also subject to a biphasic dose-response pattern which is characterized by biostimulation within a narrow band, where energy doses below a threshold fail to elicit a response and energy doses above a threshold produce a bioinhibition response.

Perhaps the most important outstanding question facing PBM is that of the mechanism of action which is still not fully understood. It is generally accepted that the primary mechanism of activation is that of mitochondrial metabolic energy pathway, and specifically the role of the chromophore CcO. However there are key observations that indicate this picture is incomplete. For example, in some studies the effects of PBM have not been accompanied by a measurable activation of CcO. Perhaps a more important observation relates to the tissue-penetration characteristics of photonic energy. Given the architecture of the dermal layers and dynamics of light-tissue interactions, most externally-applied photonic energy is absorbed in the first 2 mm of skin. With LED devices it has been shown the virtually all photonic energy is absorbed at this level. Providing energy penetration of just 1-2% of source output power to the depth of the neocortex (2-3 cm), for example, requires the use of a pulsed laser operating at a power output exceeding the high-end of most PBM-based applications. Yet, while the light from LED sources is absorbed in the outer layers of the skin, neurological studies have shown correlated EEG responses in the brain, photochemical changes in brain tissue, and positive clinical results.

In the LED example, if very little photonic energy from the source reaches the brain, how are the results to be explained? An increasingly plausible explanation is that a secondary alternative and/or complementary activation pathway may exist. A number of researchers are now postulating that the water that pervades biological tissues may be this pathway. Indeed water may play a more central role in the energy dynamics and mediation biomolecular interactions, as well as long-range coordination and communication of biological function than previously imagined.

The conventional view of water's role in biological systems is that it is a hydration and thermal regulation medium within which biomolecular interactions take place. Essentially water has been viewed as an inert solvent; a suspension medium in which macromolecules move about and waste materials are transported away. It is becoming increasingly evident that this view is limited in perspective for a number or reasons. To begin, water in and of itself is an extraordinary and remarkable molecule, possessing a unique range of anomalous phase, density, physical, material, and thermodynamic properties—more than any other chemical compound. Water's complex and anomalous character is due to its propensity for intermolecular hydrogen bonding, enabling dynamic morphological flexibility in forming supramolecular structure and networks. This morphology in turn enables efficient transfer of energy (electrons and protons) and information (resonant phase and frequency). These characteristics have profound implications relative to biological function and life itself. In fact, life as we understand it cannot exist on earth and in the universe without water.

Experimental studies have shown that the molecular and energy dynamics of water at interfaces and in confined nanospaces are shown to be different than in that of free ‘bulk’ water, and that these differences may be explained in both classical and non-classical terms. Human beings are 70% water by volume and 99% by molar concentration, and the majority of this water exists in proximity to the biological interfaces of cell membranes and within confined spaces in and around macromolecules, microtubules, channels and other structures. Here water dynamically responds to its environment structurally and energetically, mediating and driving a variety of critical biological functions. For instance, hydration layers around protein surfaces selectively respond to heterogeneous region- and structure-specific variations, guiding and mediating protein folding, structure, and stability. Water also actively guides and mediates the intra- and intermolecular recognition of protein binding partners, including nucleic acids. Moreover, the results of experimental and MD-based simulations show that water appears to facilitate cellular energy transport by dynamically forming transient linear chains of molecules, so-called ‘water wires’ that promote directional proton transport through membrane proteins such as C-type oxidases. Of critical significance in the proton transport process which drives the synthesis of ATP, is that water may effectively gate the transfer pathway so as to prevent a short-circuit resulting from premature electron-coupled proton transfer directly to the binuclear center.

Studies suggest that hydration shell dynamics also appear to strongly influence DNA conformation wherein changes in hydration state (specifically water structure) can change the structure and function of DNA. The hydration layer imbues the DNA surface with an electric field; this coupling enables energy to be exchanged between DNA and water that may serve to redistribute excess energy and maintain thermodynamic equilibrium after the decay of excitation states. Water also plays an important role in the structure and function of cell membranes. It has been shown that water at the surface of the phospholipid bilayers that form the lamellar structure of cell membranes is ordered and possesses directional symmetry correlated with that of the lipid. Researchers have found evidence that intracellular water may possess unique properties that support the critical process of ATP synthesis. An experimental study involving yeast found that dipolar relaxation of water within cytosol oscillates with glycolysis and in phase with ATP concentrations, indicating that water is bi-directionally coupled with core cellular metabolic processes. The phenomenon was found to be scale-invariant, showing coherently coupled oscillations across an ensemble of cells. Another study showed that mammalian cytoplasm behaves like a hydrogel and may detect and modulate cell volume perturbations caused by osmolality changes.

Additionally, some researchers have proposed that water may play a key role in long-range signaling across natural biological structures found in all eukaryotic cells such as membrane nanotubes, microtubules and mitochondria, and that the speed and efficiency of this information transfer may be enhanced by collective quantum coherent states among populations of ordered water molecules. One theoretical study postulated that mitochondria within membrane nanotubes may form self-organized connected network superstructures where ordered water inside these mitochondria and within their membranes facilitates a dynamic coupling leading to coherent oscillations across the network. These networks would then enable the exchange of energy and information between cells. Theoretical studies of brain microtubules have yielded the hypothesis that the collective dynamics of water confined within these structures is able to achieve QED-coherent condensed matter states of superradiance predicted under quantum field theory. Such quantum coherent states are then thought to be able to support efficient long-range transfer of energy and information. These ideas are supported by an experimental study involving the unique properties of mono-molecular water channels inside neuron-extracted microtubules, showing they integrated proteins within the microtubule walls through resonant oscillations effectively governing the electronic and optical properties of the structure.

Water's morphological flexibility makes it a versatile oscillator; readily interacting with others of its own species and with complex macromolecules such as proteins and DNA in near resonant modes. Water is a chromophore—photo-acceptor and donor—with a particularly strong absorption coefficient profile in the infrared spectral range (Beer-Lambert law). Absorption of infrared light has been shown to substantially alter both the energetic and structural dynamics of water. Once water has absorbed photonic energy in its liquid phase, it is known to be an ultrafast conductor of resonant and near-resonant vibrational excitation energy.

In PBM therapy, while IR photonic energy may indeed be absorbed by the Cytochrome c Oxidase protein complex of the inner mitochondrial membrane, it is absorbed more broadly by the water chromophore existing in abundance throughout the intra- and extracellular space of the target tissue volume. Greater concentrations of water within tissues correlate with higher absorption of infrared radiant energy. Absorbed photonic energy in water molecules is electrochemically transduced into other energy forms including vibronic and electronic excitation states in accordance with the Stark-Einstein Photoelectrochemical Equivalence Law. Absorbed energy propagates and may be further transformed by the structural characteristics of the biomolecules it interacts with (proteins, DNA, Lipids, etc.), as well as induced fields from cellular metabolism. Many researchers are beginning to consider the possibility that the water oscillator mechanism may supplement or perhaps precede the CcO pathway for infrared light-induced biomodulatory activation.

Given the multifractal dynamics of living organisms such as that of the human body, how does excitation energy progress across this complex landscape and find its way to tissues exhibiting degraded function due to injury, disease, and disorder? Experimental and MD simulation studies have shown that efficient quantum coherent energy transport processes are paradoxically enhanced by environmental noise and disorder, such as that present in the wet, noising and thermodynamically fluctuating environment of the human body. Santana-Blank and colleagues have investigated the proto-induced water oscillator question for more than two decades, and have hypothesized that the apparent biomodulating effects likely relate to correlations among water absorption coefficients, light spectral bands, the action spectra of biological tissues. The versatile H-bonding characteristics of the water molecule allow it to establish both harmonic and anharmonic resonance at a number of wavelength intervals in the infrared spectrum. These resonances are able to match peak intervals of the biological action spectra, enabling chaotic systems such as the human body to be in resonance while energy is transferred among nonlinear molecules with different vibrational modes. Rapid energy transport processes are enabled by ultrafast migration of protons in H-bonded networks of water molecules.

Santana-Blank postulates that propagating excitation energy may then be selectively absorbed by tissues expressing redox injury potential in accordance with the second law of thermodynamics and Onsanger's theory of reciprocal relations. Tissues expressing degraded function such as cancerous tumors exhibit positive oxidation-reduction potentials (electron-deficiency) and are thus receptive to photo-induced excitation energy available within the water of extra- and intracellular spaces. Because the water oscillator is a ubiquitous molecular species in living organisms, this mechanism may act upon all cellular structure and function, and with effects that vary based upon input frequency, fluence, exposure, and tissue type.

### SUMMARY OF THE DISCLOSURE

A method of inducing biomodulation effects within a living subject is provided, comprising the steps of positioning a target tissue volume of the living subject within a fluid volume such that the target tissue volume is generally aligned with a light-emitting system, irradiating the fluid volume with the light-emitting system to produce an elevated molecular excitation state within the fluid volume while the target tissue volume is disposed within the fluid volume, and exposing the target tissue volume to the elevated molecular excitation state within the fluid volume via coherent resonance energy transfer to affect biomodulation within the target tissue volume.

As described herein the method can further include an isocenter of a radial beam plane of the light-emitting system is configured to be generally aligned with the target tissue volume.

It is proposed that the light-emitting system comprises an array of inward directed light-emitting devices. The light-emitting system can comprise, for example, at least one light-emitting diode, or at least one laser.

The living subject can be fully or partially immersed within the fluid volume during biomodulation therapy. For example, the method can include fully immersing the living subject within the fluid volume, immersing at least a portion of the living subject within the fluid volume, immersing a limb of the living subject within the fluid volume, immersing a head of the living subject within the fluid volume, or immersing a torso of the living subject within the fluid volume.

A biomodulation system is also provided, comprising a fluid container sized and configured to hold a fluid and a target tissue volume of a living subject, a light-emitting system configured to irradiate the fluid of the fluid container and to irradiate the target tissue volume of the living subject, and a computer controller configured to control operating parameters of the light-emitting system to produce an elevated molecular excitation state within the fluid to affect biomodulation within the target tissue volume via coherent resonance energy transfer.

As described herein the system can further include that an isocenter of a radial beam plane of the light-emitting system is configured to be generally aligned with the target tissue volume.

It is proposed that the light-emitting system comprises an array of inward directed light-emitting devices. The light-emitting system can comprise, for example, at least one light-emitting diode, or at least one laser.

The living subject can be fully or partially immersed within the fluid volume during biomodulation therapy. For example, the system can include a fluid container for fully immersing the living subject within the fluid, immersing at least a portion of the living subject within the fluid, immersing a limb of the living subject within the fluid, immersing a head of the living subject within the fluid, or immersing a torso of the living subject within the fluid.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1B show one embodiment of a biomodulation system.

FIG. 2 shows another embodiment of a fluid container of a biomodulation system.

FIG. 3 shows one embodiment of a light-emitting apparatus of a biomodulation system.

FIG. 4 illustrates a fluid management system of a biomodulation system, including a storage tank and a fluid management system.

FIG. 5 is a schematic diagram of a biomodulation system.

### DETAILED DESCRIPTION

The present invention seeks to exploit the water oscillator (resonance) energy pathway as a potentially more efficient and effective alternative to the photonic (radiant) energy pathway. This approach employs water as the dominant molecular species within living things and, as such, has the potential to substantially mitigate the challenges and limitations of transmitting photonic energy beyond superficial depths within living tissues.

Considering the growing body of evidence that disruptions water in the structure and energy dynamics can in some way drive disease pathology, as well as the idea that those dynamics may be re-established by ‘charging’ water with infrared photonic energy, then it would appear intuitive that water represents a potential therapeutic pathway in and of itself. Biologically active water existing in abundance throughout the nano-spaces of the living organism may be viewed as a kind of resonant transceiver of energy and information supporting biomolecular structure and stability, biochemical interactions, as well as helping to maintain correlated biological activity across the entire organism. This transceiver may also serve as an important mechanism by which the organism interacts with its environment and other organisms within its ecosystem. If so, this mechanism may present the opportunity to tap into a channel of bio-information communication and energy transduction. That is, it may be possible to access and exploit water as a bio-communication pathway to impart specific, targeted phase and frequency information into the living system. The water oscillator pathway may serve as a primary therapeutic modality; or as an adjuvant to conventional therapeutic methods.

As a treatment pathway, the immersion method would seek to exploit the water oscillator as a potentially more reliable and effective delivery pathway for photo-induced excitation energy. It may be that energy propagating as resonant excitation energy across networks of water molecules partially mitigates the challenges and potential risks associated with light-tissue interactions. Such resonance energy may propagate more effectively and to deeper levels than photonic energy which is scattered or absorbed by the multitude of biological chromophores that exist within the complex architecture of the skin and subcutaneous tissues.

Under the immersion method, specific wavelengths of coherent, infrared low-level light would serve as an input energy source to a water volume which functions as a light energy-harvesting medium. In accordance with the Stark-Einstein Law (photochemical equivalence), the light energy will be absorbed by the molecules along the beam path and transduced into heat and resonance energy. This excitation energy propagates through the water volume along the excitation path via the resonance energy transfer mechanism. A body immersed within the liquid water volume, being composed largely of water, may after some interval of exposure acquire this energy from the resonant ‘system bath’. The exposure interval is dependent upon a number of variables including the input wavelength, fluence, pulse frequency and duration, fluid temperature, and target tissue volume location and physiology. Energy acquired from the bath may then traverse the biological water within tissues, again, via resonance energy transfer dynamics. This propagating excitation energy may then be selectively absorbed by sites expressing injury associated with disease and disorder, thus activating and/or supplementing cellular metabolic energy pathways in reparative and regenerative processes.

It is an objective of this disclosure to provide systems and methods for curative, adjuvant, palliative and neoadjuvant therapeutic treatment of complex physiological diseases and disorders including oncological, immunological, neurological, ophthalmological, dermatological, musculoskeletal, cardiological, pulmonological, genetics and other conditions.

It is an objective of this disclosure to provide systems and methods for promote healing from injuries including those from surgical procedures, neurotoxic, cytotoxic and genotoxic agents, blunt force traumas, concussive forces, punctures, cuts, sprains, tears, burns, frostbite, radiation, accidents and other sources.

It is an objective of this disclosure to provide systems and methods for treating psychological disorders post-traumatic stress, anxiety, behavior, and other conditions.

It is an objective of this disclosure to provide systems and methods for treating prophylactically to promote protective physiological processes related to immunological, neurological, ophthalmological, dermatological, musculoskeletal, cardiological, pulmonological, genetic and other areas of health and well-being.

It is an objective of this disclosure to provide systems and methods for studying the coherent resonance energy transfer phenomenon generally, and specifically with respect to its interactions with biological tissues and living systems.

It is an objective of this disclosure to provide systems and methods for developing biomimetic applications employing photo-induced resonance energy transfer across fluid mediums.

The conceptual foundations underlying this novel approach to biomodulation include the known phenomenon of light-matter interactions, light-tissue interactions, photoelectrochemical processes, coherent resonance energy transfer (CRET), quantum electrodynamic (QED) coherence in condensed matter, photobiomodulation, and the electron transport chain in the cells of living organisms.

The phenomenon of resonance energy transfer within fluid, and liquid water in particular, plays a central role within the context of this disclosure. It is an objective of this disclosure to provide systems and methods that facilitate and enhance the directional transfer of biomodulating energy between low-level light sources and living systems, both of which are immersed within a fluid medium. The light produced by the light-emitting sources is absorbed by a fluid and transformed into other forms of energy by well-known photoelectrochemical processes. A significant portion of the light energy is transformed to resonance energy and propagated across the fluid medium by means of coherent resonance energy transfer to a living system. The living system, comprised primarily of water molecules bound to varying degrees with resonant biomolecules, acquires biomodulating energy from the resonant ‘bath’ that is then transferred and selectively ‘absorbed’ by tissues within a target volume. The present disclosure incorporates a specialized geometric configuration of light-emitting devices operating in conjunction with a fluid immersion container and fluid management system to communicate biomodulating information to and into living organisms. In some embodiments, the fluid management system can enhance or modify the fluid volume to increase the energy transfer dynamics of the fluid volume.

Systems and methods are provided herein for positioning a living organism within a fluid container containing an aqueous medium, in proximal relationship to a geometric configuration of light-emitting devices, and creating a radial beam plane of photo-induced coherent resonance energy within that medium such that the isocenter of the radial plane is consistent with a target volume within the living organism. Computer algorithms can be implemented to control the specific operating parameters in combination, including power density, energy density, pulse frequency, wavelength, irradiation time and others. Treatment protocols can be pre-programmed based upon known conditions and treatment objectives, as well as created ad-hoc in accordance individual factors related to the patient condition.

According to the present disclosure, the focus of biomodulation using low-level light emitting devices and a fluid immersion pathway is the coherent resonance energy transfer of excitation energy to resonant biomolecules in living systems. To date, there has been no published research or prior art citations relative to the combined use of light-emitting devices, a fluid immersion pathway, and coherent resonant energy transfer to create and facilitate biomodulation effects within living organisms. The present disclosure provides an alternative approach to photobiomodulation which embraces the idea that, as biological entities composed largely of water, living organisms can interact with a surrounding resonant aqueous medium with a high level of energy efficiency. Drawing upon the concepts of CRET and the growing evidence of ordered water molecules throughout the tissues and nano-spaces of living organisms, the idea employing an external aqueous medium as a communication pathway to impart biomodulating energy into living things appears viable.

As described in the related U.S. application Ser. No. 14/930,516, the systems and methods described herein can create a medium encompassing the living organism and the surrounding water volume, enabling the exchange of energy within a highly resonate environment. This medium is in essence a technology-enabled bio-communication pathway, and is bi-directional. The bi-directional communication presents the capability to pursue a number of research, diagnostic, and therapeutic applications.

The present disclosure describes the use of a biomodulation apparatus including an array of light-emitting devices, a water-proof housing, a water-proof harness for system monitoring and control wiring, and a temperature control function. This biomodulation apparatus is specifically designed to induce photoexcitation within the molecular species present within a fluid volume. The fluid volume can be contained within a specially designed fluid container suitable for use with the light-emitting apparatus. A fluid management system can also be included.

The biomodulation apparatus can include a computerized control system configured to control photoexcitation of the fluid volume both with and without one or more living organisms present within the fluid volume. The photoexcitation parameters can be pre-programmed or configured on-the-fly, including: irradiance (W/ [symbol] cm [symbol] \hat{ }2), energy density (J/ [symbol] cm [symbol] \hat{ }2), pulse frequency (Hz), pulse width (s), peak power (W), wavelength (nm), irradiation time (s), treatment interval (D/W/M), and others. Treatment session execution data can be captured and stored.

Research and practical use of this apparatus and method can increase understanding of the treatment regime response characteristics and dynamics as related to specific conditions in various living organisms. This knowledge can further enable the development of integrated databases for treatment planning, aiding practitioners using this system and method.

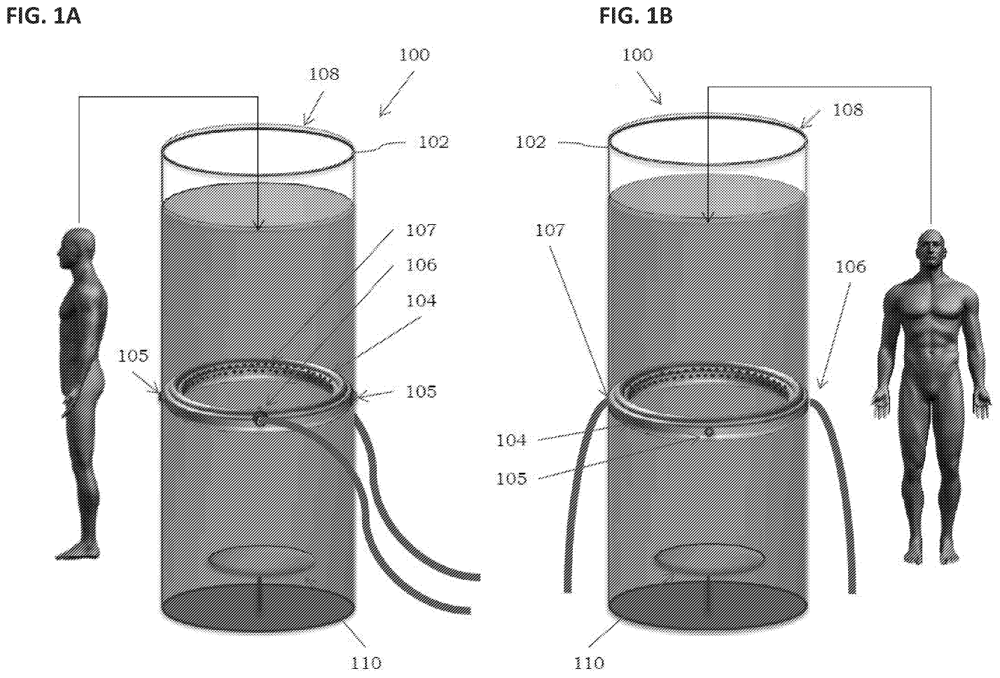

FIGS. 1A and 1B illustrate, respectively, front and side views of a biomodulation system 100 configured to induce a radial plane of photoexcitation around a living organism, such as a human being. The biomodulation system 100 can comprise, generally, a fluid container 102, a light-emitting system 104, and access ports for a system wiring harness 106. The light-emitting array assembly 104 further includes an array of light-emitting devices, one or more access ports for temperature control ducts 107, and one or more array mounts 105.

The fluid container can generally be sized and configured to contain a fluid, a light-emitting system and a living organism to be treated. The embodiment shown in FIGS. 1A and 1B includes a cylindrical fluid container sized for evaluating adult humans, but fluid containers of other sizes can be implemented for treating extremities as well as smaller/larger living organisms.

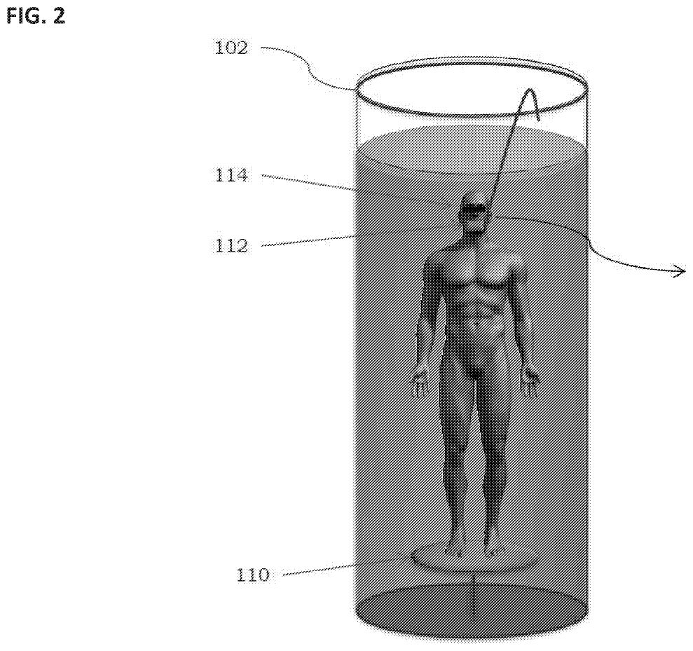

As shown in FIGS. 1A-1B and FIG. 2, the fluid container 102 can comprise a transparent material such as acrylic glass (Poly Methyl Methacrylate) or glass. A transparent material is important for enabling the patient positioning via a set of cameras covering the x, y, and z axis of the fluid container. Transparent materials also provide for lower patient stress and the ability of system operators to visually monitor and communicate with patients before, during, and after therapeutic treatment.

In the illustrative embodiment, a human subject is fully immersed in the fluid container 102. In one embodiment, the subject can be immersed standing upright or sitting in the fluid. In one specific embodiment, the container can be cylindrical in shape having dimensions of roughly 2-3 m in height, by 1-1.5 m in width, and a wall thickness of 1-2 cm. In other embodiments, the fluid container can be horizontally positioned to enable biomodulation while the human subject is in the prone position. In other embodiments, the fluid container can comprise various additional shapes. For example, the fluid container 102 can be rectangular or triangular in shape. For other types of living organisms, dimensional variations of rectangular, cylindrical, or spherical shapes may be used.

The fluid container can be filled with a fluid, such as water, having a temperature that is comfortable to the human subject (e.g., approximately room temperature water), but not of sufficient temperature to produce significant convective motion. In some embodiments, purified water can be used. Water can be desirable for use with the biomodulation due to its unique properties and capacity for resonant interaction with biological water and other biomolecules. Other fluid mixtures containing a combination of water and other molecular compounds with chemically and biologically-suitable characteristics can be used, such as trace amounts of NaCl. In some embodiments, the fluid container can be filled with a fluid from a fluid management system (described below) with a fluid specifically conditioned to maintain an enhanced-level of energy transfer within the fluid volume.

In one embodiment, the fluid container can be accessed through an open top portion 108. The human subject can be lowered into the fluid through the top portion and stand upon or be supported by a platform 110 positioned near the bottom of the container. The platform 110 can comprise the same material as the fluid container, such as an acrylic glass, or be comprised of aluminum-alloy structural components. The fluid level in the fluid container and the platform height can be adjusted in accordance with the area of the organism to be irradiated with the radial beam plane. The platform may also be designed to be raised or lowered slowly during treatment in order to distribute excitation energy across a target tissue volume with vertical dimensions exceeding the width of the radial beam plane. In other embodiments, the light-emitting system can be designed to be raised and lowered during treatment.

With biomodulation treatment of the head, the subject must be fully submerged in the fluid, with sufficient fluid coverage above the head. For example, the platform can be adjusted to provide at least 30 cm above the head of the subject, ensuring the treatment can substantially address the top of the cranial vault and scalp. For treatment of targeted body regions below the head and neck, it may not be necessary to fully submerge the subject. As shown in FIG. 2, when the subject is fully submerged in the fluid, a breathing apparatus 112 such as a snorkel or breathable-air line can be used to allow the subject to breath. Eye protection 114 is also used when continuous wave and pulsed laser devices are employed within the light-emitting array.

Still referring to FIGS. 1A and 1B, the light-emitting system 104 can be fitting inside the fluid container 102 and can comprise a light-emitting array including a plurality of light emitting sources, a water-proof housing, power and control harness and cooling duct access ports, and mount ports in the wall of the fluid container. The light-emitting devices used may include intense pulsed light, LEDs, Diode Lasers, Fiber Lasers, Optical Parametric Oscillators (OPO) and others. The light-emitting system assembly can be configured to generate a field of excitation energy within the fluid volume of the fluid container. In other embodiments, the light-emitting system can be designed to fit outside of the fluid container, with and without access ports for the apertures of the light-emitting devices of the light-emitting array.

The dimensions of the fluid container and biomodulation system can vary based upon the need to evaluate whole-body versus body extremities, as well as the types of organisms to be evaluated. For smaller organisms or extremities, the dimensions of the fluid container can allow for smaller fluid containers and light-emitting assemblies to be employed. Larger organisms, and therefore larger fluid containers, may require modified biomodulation system configurations. For example, a fluid container designed for human subjects may need to accommodate a vertical standing or sitting position, in addition to a horizontal prone position. To accommodate both positions, the biomodulation system can include a frame configured to be rotated and elevated. In order to provide maximum flexibility for handling both vertical and horizontally-aligned treatment sequences, the platform within the container can be designed to raise or lower a patient before or during treatment procedures. In some embodiments, the fluid container is emptied during this rotation process to minimize the stress loads involved this operation.

The biomodulation system configurations described herein create a medium in which the subject, as a living system comprised of tissues containing primarily water molecules, is immersed within a fluid volume that can be enhanced to optimize its resonance energy transfer dynamics. In the context of this disclosure, resonance energy transfer in a bulk fluid volume is induced when exposed to the light-emitting system operating at specific wavelengths, pulse frequencies and beam energy parameters. The systems and methods described herein enable resonant interplay between the water molecules in the fluid volume and living organisms in which phase and frequency information is exchanged. Resonant energy transferred from the bulk fluid volume into the subject changes vibrational and electronic state of the water molecules therein. These resonance energy-induced changes in the living system represent an enhanced degree of coherence (and thus structure) of biological water from that of the disease or disorder conditions.

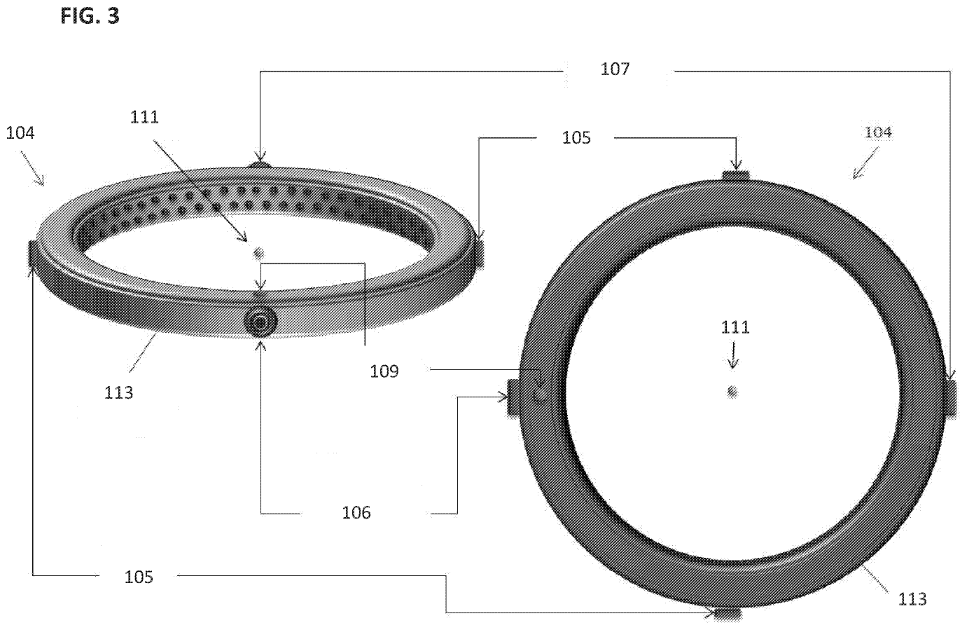

Referring now to FIG. 3, the biomodulation system 100 can comprise a light-emitting array assembly 104 that includes an array of light-emitting devices, one or more access ports for system control and monitoring harnesses 106, one or more access ports for temperature control ducts 107, one or more array mounts 105, a system status indicator 109, and a water-tight housing 113. In one embodiment, the light-emitting array 104 may be comprised of infrared lasers operating at specific monochromatic spectral wavelengths correlated to the absorption peaks of water and other chromophores. In other embodiments, the light-emitting array 104 may be comprised of other light-emitting devices including intense pulsed light, LED, Optical Parametric Oscillators (OPO) and others.

Still referring to FIG. 3, the center point of the light-emitting array 104 may be referred to as the radial beam isocenter 111. This point represents the point of focus for each inward-directed light-emitting device within the array assembly, and is the convergence point of resonance energy. In one embodiment this point is located in the exact center of the radial beam plane, equidistant from all beam sources, and is the isocenter of the resonance energy formed by the convergent radial beams. In some applications of this invention, the procedural objective is to position target tissue volume of living subject at this point, similar to the procedure employed in conventional radiation therapy. In other embodiments, the beam isocenter may be focused upon other points within the fluid volume.

The fluid container and light-emitting system dimensions can be matched to the dimensions of the living organism such that a distance of 50-70 cm is maintained between the light-emitting array and the outer surface of the subject. This positioning approach is designed to ensure that sufficient photo-induced excitation energy and CRET is present in the bulk water volume to affect biomodulation within the living subject.

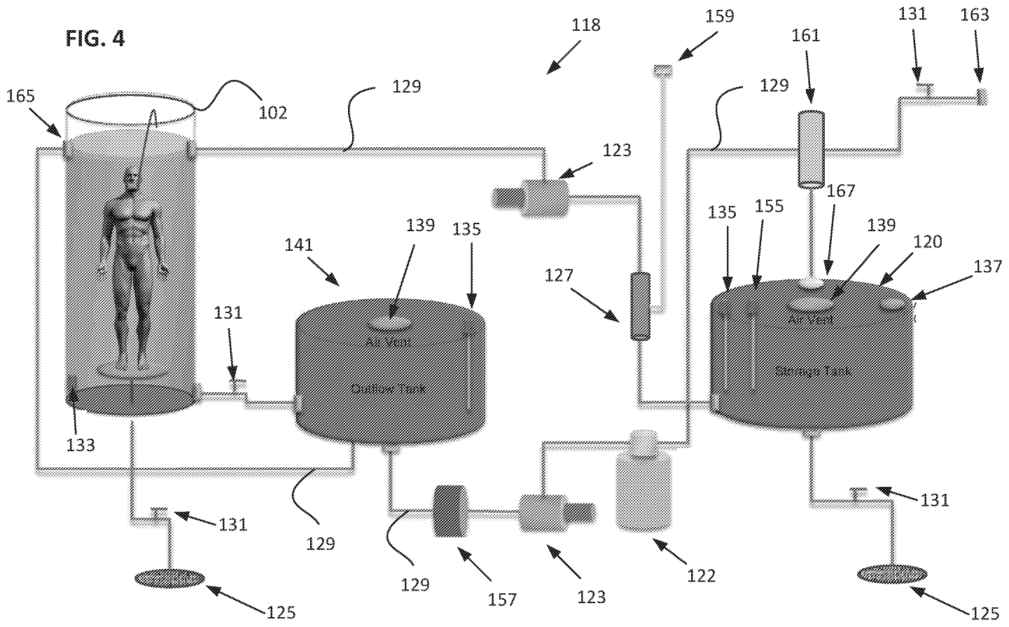

Referring now to FIG. 4 (and FIG. 4 in U.S. application Ser. No. 14/930,516), the biomodulation system 100 can further comprise a fluid management system 118 that includes a storage tank 120, a filtration system 122, one or more pumps 123 configured to pump fluid from the storage tank 120 into and out of the fluid container 102 via lines 129 and valves 131. The pumps 123 can optionally pump fluid out of the fluid container into one or more drains 125 and/or the storage tank 120 or outflow tank 141, and can also be configured to maintain the fluid level in the fluid container 102. Fluid in the fluid container can be rapidly drained to drain 125 through a valve 131 in the event that the fluid volume is contaminated or needs to be drained quickly. The storage tank 120 can be sized and configured to hold a large enough volume of fluid to fill the fluid container 102 during or prior to biomodulation. In some embodiments, the storage tank can hold enough fluid to fill the fluid container several times over.

The fluid management system 118 can further comprise a temperature control system 127 configured to control the temperature of the fluid as it is delivered to the fluid container, including either heating or cooling the fluid. The temperature control system can be powered by, for example, a natural gas source 159.

Additional features can be included in the fluid management system, such as a temperature sensor 133 in the fluid container, a level sensor 135 in the storage tank and outflow tanks, a PH sensor 155 in the storage tank, an access cap 137 in the storage tank for visual inspection of the storage tank, and air vents 139 in the storage and outflow tanks for venting the tanks. The fluid container 102 can optionally include an overflow outlet 165 to prevent the container from being overfilled with fluid.

Referring still to FIG. 4, water can be introduced into the system from water source 163 which may be tap water or a rainwater-capture cistern. These sources typically contain solutes that may encourage resonance energy transfer dynamics within the water volume. The valve 131 can be controllable from a system control console or, alternatively, may be manually controlled.

Input water can be funneled through a water conditioning unit 161. This water conditioning unit can be any water conditioning unit known in the art, such as an acrylic glass cylinder filled with glass or quartz crystal spheres, and can be positioned vertically above the storage inlet 167. The water can be allowed to percolate from the top down of the water conditioning unit and into the storage tank 120. In some embodiments, the water conditioning unit may also contain solid tablets of non-aqueous compounds, such as NaCl or KCl. This enables the water conditioning unit of the fluid management system to add trace amounts of non-aqueous compounds to the water as it is introduced into the fluid management system. This water conditioning unit is designed to promote molecular ordering, thereby enhancing the energy transfer dynamics of the fluid delivered to the fluid container.

Fluid in the fluid management system can be recycled by emptying the fluid volume from the fluid container into the outflow tank 141 through valve 131. The fluid can be pumped from the outflow tank through demagnetizer unit 157 to effectively de-structure the water and thereby remove any residual EMF/bio-information imprinting contained within the water. The de-imprinted water can flow through filtration system 122 which can contain levels of various materials that may include gravel, sand, and/or charcoal to filter and/or purify the fluid. The filtered fluid can be pumped back into the water conditioning unit 161 with pump(s) 123, where it can be reconditioned and transferred into the storage tank.

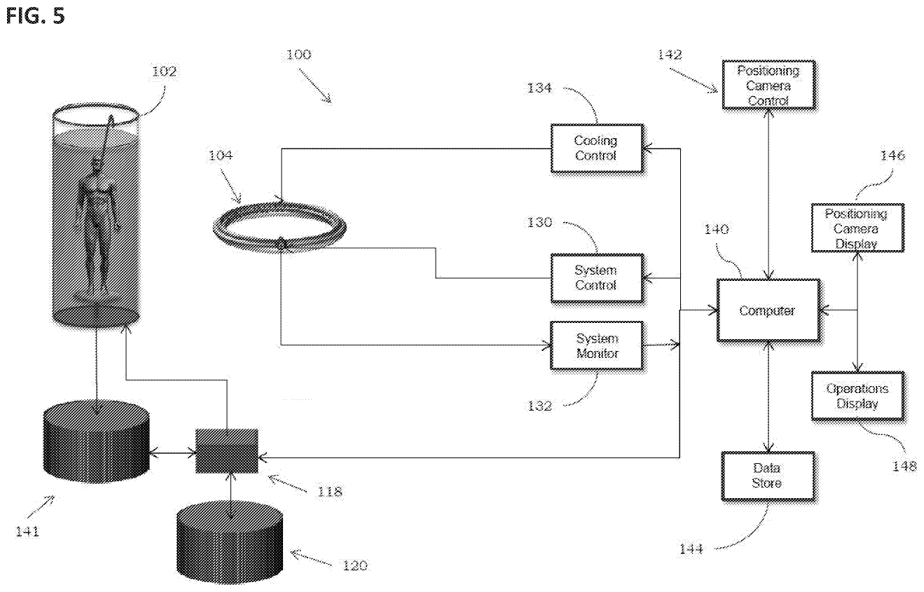

FIG. 5 is a schematic diagram illustrating additional details of the biomodulation system 100, including fluid container 102, light-emitting array 104, fluid management system 118, storage tank 120, and overflow tank 141. The light-emitting array 104 can include a plurality of light-emitting devices, power, control, and system status wiring, and temperature control ducts. The light-emitting array can be controlled by a system control 130 which controls power and operational parameters for the light-emitting devices. The light emitting array 104 operating status can be monitored by a system monitor 132 which monitors the functional status of light-emitting devices, operating temperature, and other telemetry. The temperature control 134 works with data produced by the system monitor 132 to maintain operating temperatures within target ranges.

Overall control of the biomodulation system 100 can be handled by computing system or computer 140. The computer 140 can be a remote computer system configured to control all aspects of the biomodulation system 100, including control of the light-emitting array and control of the fluid management system 118 including fluid management of the fluid container 102. The computer 140 can include hardware and software configured to control every aspect of the light-emitting array system, including executing the specific output power, pulse sequences, wavelength, energy density and other parameters necessary to produce biomodulation effects within the target tissue volume of the subject.

The computer 140 can be located in a control room of the treatment facility, enabling operators to monitor and initiate control actions from that location. In some embodiments, the computer 140 can be operatively coupled (e.g., direct electrical connection, or a wireless connection such as WiFi, Bluetooth, cellular, etc.) to the light-emitting array 104 and/or the fluid management system 118. In some embodiments, a wireless connection between the computer and the light-emitting array may be undesirable, and thus a direct electrical connection may be preferred. This configuration enables imaging control, fluid level control, as well as the ability to empty the fluid container and maintain desired water conditions between uses, or in the case of an alternative embodiment, to reposition the fluid container in the horizontal position in order to treat the human subject in the prone position.

One or more cameras can be located on or near the fluid container 102 to assist system technicians in positioning patients within the fluid volume. The computer 140 can run the patient positioning camera control system 142 comprised of cameras that can be configured to ensure that the patient is properly positioned relative to the radial beam plane of the light-emitting area. The cameras produce and capture live video on the patient positioning camera display 146. The camera(s) may also provide remote supervision or observation of the biomodulation process. In some embodiments, the computer 140 can further be configured to provide data storage 144 for all the operating data and video gathered during treatment process, and can provide display 148 relating to any and all operating parameters of the system during use. The graphical and operations displays can include an input device, such as a mouse, keyboard, trackpad, and/or graphical user interface (GUI) such as a touchscreen to allow a user to interact with the computer 140 to initiate and manage the treatment process, fluid management, monitoring of the status of various sensors.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Various modifications to the above embodiments will be readily apparent to those skilled in the art, and the generic principles defined herein may be applied to other embodiments without departing from the spirit or scope of the invention. Thus, it is intended that the scope of the present invention herein disclosed should not be limited by the particular disclosed embodiments described above, but should be determined only by a fair reading of the claims that follow.

In particular, materials and manufacturing techniques may be employed as within the level of those with skill in the relevant art. Furthermore, reference to a singular item, includes the possibility that there are plural of the same items present. More specifically, as used herein and in the appended claims, the singular forms “a,” “and,” “said,” and “the” include plural referents unless the context clearly dictates otherwise. As used herein, unless explicitly stated otherwise, the term “or” is inclusive of all presented alternatives, and means essentially the same as the commonly used phrase “and/or.” Thus, for example the phrase “A or B may be blue” may mean any of the following: A alone is blue, B alone is blue, both A and B are blue, and A, B and C are blue. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Various modifications to the above embodiments will be readily apparent to those skilled in the art, and the generic principles defined herein may be applied to other embodiments without departing from the spirit or scope of the invention. Thus, it is intended that the scope of the present invention herein disclosed should not be limited by the particular disclosed embodiments described above, but should be determined only by a fair reading of the claims that follow.

In particular, materials and manufacturing techniques may be employed as within the level of those with skill in the relevant art. Furthermore, reference to a singular item, includes the possibility that there are plural of the same items present. More specifically, as used herein and in the appended claims, the singular forms “a,” “and,” “said,” and “the” include plural referents unless the context clearly dictates otherwise. As used herein, unless explicitly stated otherwise, the term “or” is inclusive of all presented alternatives, and means essentially the same as the commonly used phrase “and/or.” Thus, for example the phrase “A or B may be blue” may mean any of the following: A alone is blue, B alone is blue, both A and B are blue, and A, B and C are blue. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs.

What is claimed is:

1. A method of inducing biomodulation effects within a living subject, comprising the steps of:
positioning a target tissue volume of the living subject within a fluid volume such that the target tissue volume is generally aligned with a light-emitting system;
irradiating the fluid volume with the light-emitting system to produce an elevated molecular excitation state within the fluid volume while the target tissue volume is disposed within the fluid volume; and
exposing the target tissue volume to the elevated molecular excitation state within the fluid volume via coherent resonance energy transfer to affect biomodulation within the target tissue volume.

2. The method of claim 1, wherein an isocenter of a radial beam plane of the light-emitting system is configured to be generally aligned with the target tissue volume.

3. The method of claim 1, wherein the light-emitting system comprises an array of inward directed light-emitting devices.

4. The method of claim 1, wherein the light-emitting system comprises at least one light-emitting diode.

5. The method of claim 1, wherein the light-emitting system comprises at least one laser.

6. The method of claim 1, wherein positioning the target tissue volume comprises fully immersing the living subject within the fluid volume.

7. The method of claim 1, wherein positioning the target tissue volume comprises immersing at least a portion of the living subject within the fluid volume.

8. The method of claim 1, wherein positioning the target tissue volume comprises immersing a limb of the living subject within the fluid volume.

9. The method of claim 1, wherein positioning the target tissue volume comprises immersing a head of the living subject within the fluid volume.

10. The method of claim 1, wherein positioning the target tissue volume comprises immersing a torso of the living subject within the fluid volume.

11. A biomodulation system, comprising:
a fluid container sized and configured to hold a fluid and a target tissue volume of a living subject;
a light-emitting system configured to irradiate the fluid of the fluid container and to irradiate the target tissue volume of the living subject; and
a computer controller configured to control operating parameters of the light-emitting system to produce an elevated molecular excitation state within the fluid to affect biomodulation within the target tissue volume via coherent resonance energy transfer.

12. The system of claim 11, wherein an isocenter of a radial beam plane of the light-emitting system is configured to be generally aligned with the target tissue volume.

13. The system of claim 11, wherein the light-emitting system comprises an array of inward directed light-emitting devices.

14. The system of claim 11, wherein the light-emitting system comprises at least one light-emitting diode.

15. The system of claim 11, wherein the light-emitting system comprises at least one laser.

16. The system of claim 11, wherein the fluid container is sized and configured to fully immerse the living subject.

17. The system of claim 11, wherein the fluid container is sized and configured to immerse at least a portion of the living subject.

18. The system of claim 11, wherein the fluid container is sized and configured to immerse a limb of the living subject.

19. The system of claim 11, wherein the fluid container is sized and configured to immerse a head of the living subject.

20. The system of claim 11, wherein the fluid container is sized and configured to immerse a torso of the living subject.
